(19) United States
(12) Patent Application Publication
(10) Pub. No.: US 2013/0225437 A1
(43) Pub. Date: Aug. 29, 2013

(54) BIOMARKERS OF CANCER

(71) Applicants: Mayumi Fujita, Englewood, CO (US); Charles A. Dinarello, Boulder, CO (US); Yuchun Luo, Denver, CO (US); William A. Robinson, Denver, CO (US); David A. Norris, Englewood, CO (US)

(72) Inventors: Mayumi Fujita, Englewood, CO (US); Charles A. Dinarello, Boulder, CO (US); Yuchun Luo, Denver, CO (US); William A. Robinson, Denver, CO (US); David A. Norris, Englewood, CO (US)

(73) Assignee: THE REGENTS OF THE UNIVERSITY OF COLORADO , a body corporation

(21) Appl. No.: 13/817,215

(22) PCT Filed: Aug. 16, 2011
(86) PCT No.: PCT/US2011/047931

§ 371 Date: Apr. 26, 2013

Related U.S. Application Data
(60) Provisional application No. 61/374,249, filed on Aug. 16, 2010.

(51) Int. Cl. C12Q 1/68; G01N 33/68

(52) U.S. Cl. CPC C12Q 1/6886; G01N 33/6869
USPC 506/9; 435/6.12; 436/501; 435/7.1

(57) ABSTRACT

Disclosed is an IL-37 biomarker of cancer and methods of using the biomarker, including methods for diagnosis of cancer, methods of determining predisposition to cancer, methods of monitoring progression/regression of cancer, methods of assessing efficacy of compositions for treating cancer, as well as other methods based on the use of this cancer biomarker.

### CROSS REFERENCE TO RELATED APPLICATION

This application claims the benefit of priority under 35 U.S.C. §119(e) to U.S. Provisional Patent Application Serial No. 61/374,249 filed Aug. 16, 2010, which is incorporated herein by reference.

### GOVERNMENT INTEREST

This invention was made with government support under grant number R02 CA125833 awarded by the National Institutes of Health. The government has certain rights in the invention.

### FIELD OF THE INVENTION

The present invention generally relates to biomarkers, methods and assay kits for the identification, monitoring and treatment of cancer patients.

### BACKGROUND OF INVENTION

Detection of cancer and initiation of the most appropriate therapy at an early stage are two major determinants of a successful anti-cancer therapy. For example, malignant melanoma is the most aggressive form of skin cancer. Melanoma occurs at a younger age than most cancers, with half of all melanomas found in people under age 57, and incidence rates are increasing. Despite recent progress in the technologies of molecular and cellular biology and immunology, melanoma remains a challenge in clinical oncology. Melanoma represents an extremely poor prognostic cancer that remains one of the most common and aggressive malignancies worldwide. The importance of early detection of melanoma cannot be overstated: with current diagnostic methods, melanoma patients are often diagnosed with end-stage cancer and have poor survival rates. Alternatively, when melanoma is found and treated early, the chances for long-term survival are excellent, and five-year survival rates for patients with early-stage (Stage I) cancer exceed 90 to 95%. In later-stage disease however, 5-year survival rates drop to less than 50%.

Therefore, there is a need for accurate methods to identify cancer in a patient, as well as its proclivity for metastases/relapse, particularly at early stages of the disease. In order to do so, it is important to identify novel biomarkers which can provide this information and facilitate therapeutic decisions for cancer patients.

### SUMMARY OF INVENTION

The present invention relates to biomarkers of cancer, methods for diagnosis of cancer, methods of determining predisposition to cancer, methods of monitoring progression/regression of cancer, methods of assessing efficacy of compositions for treating cancer, methods of screening compositions for activity in modulating biomarker of cancer, methods of treating cancer, as well as other methods based on biomarker of cancer.

In one embodiment, the invention provides a method for determining if a subject has cancer, the method comprising analyzing a biological sample from a subject to determine the level of a marker for cancer in the sample, wherein the marker is anti-inflammatory gene IL-1 family member 7 (previously “IL-1F7,” now known as IL-37), fragments thereof, successors thereof, and modified versions thereof; and comparing the level of the marker in the sample to cancer-positive and/or cancer-negative reference levels of the marker to diagnose whether the subject has cancer.

In some embodiments, the cancer is early stage cancer (i.e. stage 0 or stage I).

In some embodiments, the IL-37 marker is up-regulated compared to cancer-negative reference levels of IL-37.

In some embodiments, the cancer-positive and/or cancer-negative reference levels of the IL-37 marker are associated with a level of metastasis to a distant location or lymph nodes (metastatic cancer).

The invention also provides a method for determining patient outcome in cancer, the method comprising analyzing a biological sample from a subject to determine the level of the IL-37 marker, fragments of this marker, successors of this marker, modified versions of this marker, and combinations of this marker.

In some embodiments, the level of the IL-37 marker in the sample as compared to cancer-positive and/or cancer-negative reference levels of the marker is indicative of cancer outcome and prognosis.

In some embodiments, down-regulation of the IL-37 marker compared to cancer-negative reference levels is associated with an increase in patient survival.

In some embodiments, the biological sample is a tumor tissue, and/or a body fluid, such as blood, cultured whole blood, urine, or saliva.

In some embodiments, the cancer is a leukemia, pancreatic cancer, breast cancer, ovarian cancer, prostate cancer, lung cancer, or skin cancer. In a preferred embodiment, the cancer is melanoma.

In some embodiments, the standard level or reference range is determined according to a statistical procedure for risk prediction, such as using a Hazard ratio.

In other embodiments, the level of the IL-37 marker in the sample is detected with a reagent that specifically detects the marker, such as an antibody, an antibody derivative, an antibody fragment, and/or an aptamer.

The invention also provides a method for monitoring the progression of cancer in a subject, the method comprising measuring the expression level of the IL-37 marker in a first biological sample obtained from the subject; measuring the expression level of the IL-37 marker in a second biological sample obtained from the subject; and comparing the expression level of the IL-37 marker measured in the first sample with the level of the marker measured in the second sample.

In some embodiments, the first biological sample from the subject is obtained at a time t₀, and the second biological sample from the subject is obtained at a later time t₁. The first biological sample and the second biological sample may be obtained from the subject more than once over a range of times.

The invention further provides a method of assessing the efficacy of a treatment for cancer in a subject, the method comprising comparing the expression level of the IL-37 marker measured in a first sample obtained from the subject at a time t₀; and the level of the IL-37 marker in a second sample obtained from the subject at time t₁; wherein a change in the level of the marker in the second sample relative to the first sample is an indication that the treatment is efficacious for treating cancer in the subject.

In some embodiments, the time t₀ is before the treatment has been administered to the subject, and the time t₁ is after the treatment has been administered to the subject and in other embodiments, the comparing is repeated over a range of times. In some embodiments, the time t₀ is before the treatment has been administered to the subject, and the time t₁ is after the treatment has been administered to the subject.

The invention further provides an assay system for cancer comprising a means to detect the expression of the IL-37 marker. In some embodiments, the means to detect comprises binding ligands that specifically detect the marker. In other embodiments, the means to detect comprises binding ligands disposed on an assay surface. In some embodiments, the assay surface comprises a chip, array, or fluidity card. The binding ligands may comprise antibodies or binding fragments thereof

In other embodiments, the assay system comprises a control selected from the group consisting of information containing a predetermined control level of the marker that has been correlated with good patient outcome or information containing a predetermined control level of the marker that has been correlated with poor patient outcome, or both of the foregoing.

In other embodiments, the assay system comprises a control selected from the group consisting of information containing a predetermined control level of the marker that has been correlated with or associated with cancer subtype or information containing a predetermined control level of the marker that has been correlated with one or more stages of cancer and/or both of the foregoing.

Other features and advantages of the invention will become apparent to one of skill in the art from the following detailed description.

### BREIF DESCRIPTION OF DRAWINGS

FIG. 1 depicts immune responses to cancer cells: (A) generation of immune responses against cancer does not seem to successfully kill cancer cells; (B) soluble mediators derived from cancer cells may induce immuno-suppression by upregulating IL-37 expression in blood cells, leading to enhanced tumor growth and metastasis.

FIG. 2 shows an analysis of gene expression in blood cells from melanoma patients and normal healthy controls: (A) microarray analysis of whole blood cells that have 5-fold or greater differences between healthy controls (left 6 samples, C1-C6) and cancer (right 4 samples, M1-M4). Each line represents a single gene; (B) qRT-PCR analysis for IL-37 gene expression from 6 healthy controls and 17 cancer patients; (C) CD45+ cells from (B) broken down by stage of melanoma (N=6 from stage I, N=3 from stage II, N=3 from stage III and N=5 from stage IV) * p<0.05 compared with healthy controls, ** p<0.01 compared with healthy controls, *** p<0.001 compared with healthy controls.

FIG. 3 shows contact hypersensitivity reactions in wild type and IL-37 transgenic mice. (A) Ear swelling measured after challenge of DNFB-sensitized mice. Mice were sensitized on Day 0 and challenged on Day 5. (**p<0.01, ***p<0.001; n=5 mice per group.) (B) Histology revealed a marked reduction in the epidermal hyperplasia, dermal edema, vasodilation and dermal inflammatory infiltrates in TG (DNFB) skin. (C) CD3 T cell infiltration was less in TG (DNFB-challenged) mouse ears 48 hrs after the challenge. (D) IL-37qRT-PCR in two WT and two TG mice from skin before (left bars) and after DNFB sensitization (right bars). The middle bars are from skin taken distal from the sensitization site. IA/IE staining of epidermal sheets, 24 hrs after vehicle or DNFB application, revealed activated Langerhans cells (skin antigen-presenting cells) after DNFB application in WT mice but not in TG mice. (E) CD40 expression of epidermal Langerhans cells, 24 hours after application. ** p<0.01 compared with WT-DNFB. (F) Proliferation of CD4 or CD8 memory T cells stimulated with dendritic cells from WT mice (left) or TG mice (right).

FIG. 4 shows tissue distribution of IL-37 expression in metastatic melanoma tissues. (A-D) Formalin-fixed paraffin-embedded tissue sections of reactive lymph node (positive control; A, B) and metastatic cancer (C,D) were stained with control IgG (A,C) or anti-human IL-37 antibody (B,D). Immunoreactants were visualized with DAB: (A,B) x40, (C,D) x10.

FIG. 5 shows IL-37 gene expression levels in CD45+ blood cells in normal and healthy subjects.

FIG. 6 shows IL-37 gene expression levels in CD45+ blood cells. Blood samples showed statistically significant differences in IL-37 gene expression between healthy controls and melanoma patients.

FIG. 7 shows the difference in IL-37 gene expression levels in melanoma patients at different disease stages.

FIG. 8 shows that IL-37 gene expression levels >0.0005 can accurately predict disease progression in melanoma patients.

### DESCRIPTION OF EMBODIMENTS

The present inventors have discovered a biological marker whose presence and measurement levels are indicative of malignant cancer. The biomarker is differentially expressed in biological samples obtained from cancer patients and is compared to clinical outcomes. The levels and activities of the biomarker, along with clinical parameters, can be used as a biological marker indicative of cancer, including early stage disease (i.e. stage 0 or stage I). The invention also relates to the expression levels of the biomarker in patients with cancer that discriminate between patients having a high and low probability of survival.

As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if a biomarker measurement typically changes in the absence of a particular biological process, a constant measurement can indicate occurrence of that process.

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such marker. Unless defined otherwise, all scientific and technical terms are to be understood as having the same meaning as commonly used in the art to which they pertain. For the purposes of the present invention, the following terms are defined below.

As used herein, the term “marker” includes the IL-37 protein, gene, RNA, cDNA transcript, metabolite or small molecule fragment of the IL-37 molecule. Metabolite or small molecule means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

Marker measurements may be of the absolute values (e.g., the molar concentration of a molecule in a biological sample) or relative values (e.g., the relative concentration of two molecules in a biological sample). The quotient or product of two or more measurements also may be used as a marker. For example, some physicians use the total blood cholesterol as a marker of the risk of developing coronary artery disease, while others use the ratio of total cholesterol to HDL cholesterol.

In the invention, the marker is primarily used for diagnostic and prognostic purposes. However, it may also be used for therapeutic, drug screening and patient stratification purposes (e.g., to group patients into a number of stages or “subsets” for evaluation), as well as other purposes described herein, including evaluation the effectiveness of a cancer therapeutic.

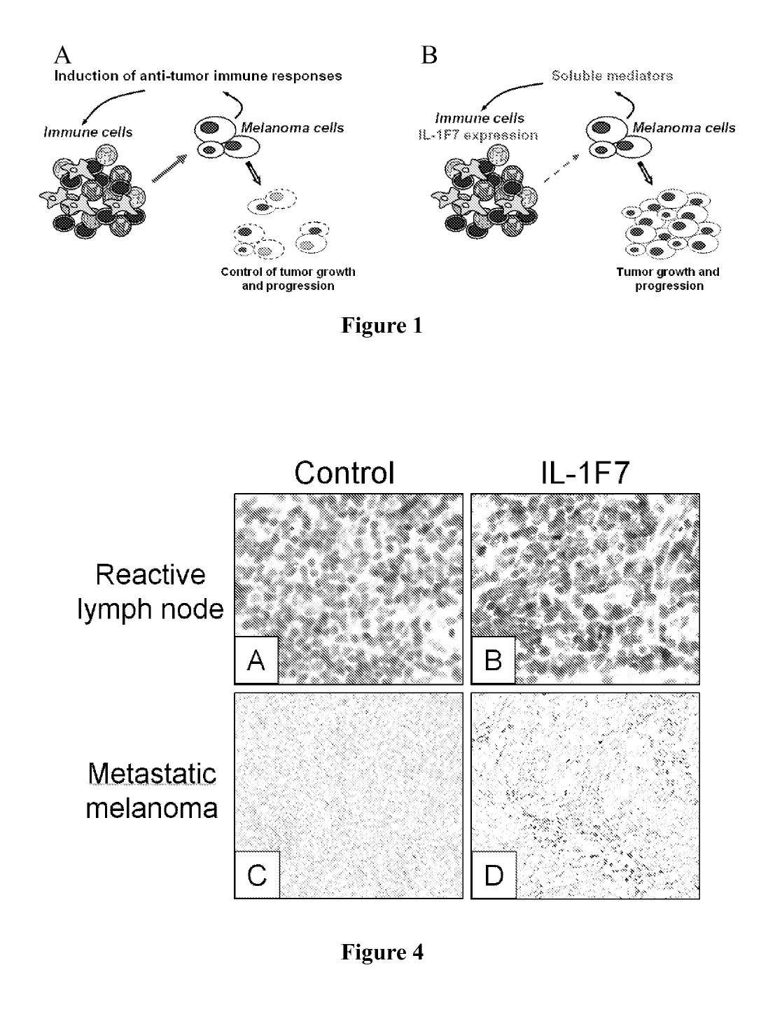

The present invention is based on the findings of a study designed to identify the mechanisms of immunomodulation and immunoevasion by cancer cells wherein an immune response to cancer cells is generated in vitro and/or in vivo, but anti-tumor responses from activated cancer-specific T lymphocytes in the tumor and blood of cancer patients do not result in any significant anti-tumor effect, presumably due to tumor-induced immunosuppression (FIG. 1A). In these studies, the present inventors identified differentially expressed pathways and genes in human cancer blood by microarray analysis. It was discovered that IL-37 is highly upregulated in blood samples taken from cancer patients. Microarray analysis of whole blood samples showed at least a five-fold difference in IL-37 expression in cancer samples over samples taken from healthy controls. Without intending to be bound by any one theory, the IL-37 expression is detected in white blood cells that are believed to have been chemically activated or physically contacted by a cancer cell and have become educated or induced to express IL-37. It is believed that IL-37 is normally expressed primarily in granulocytes and monocytes.

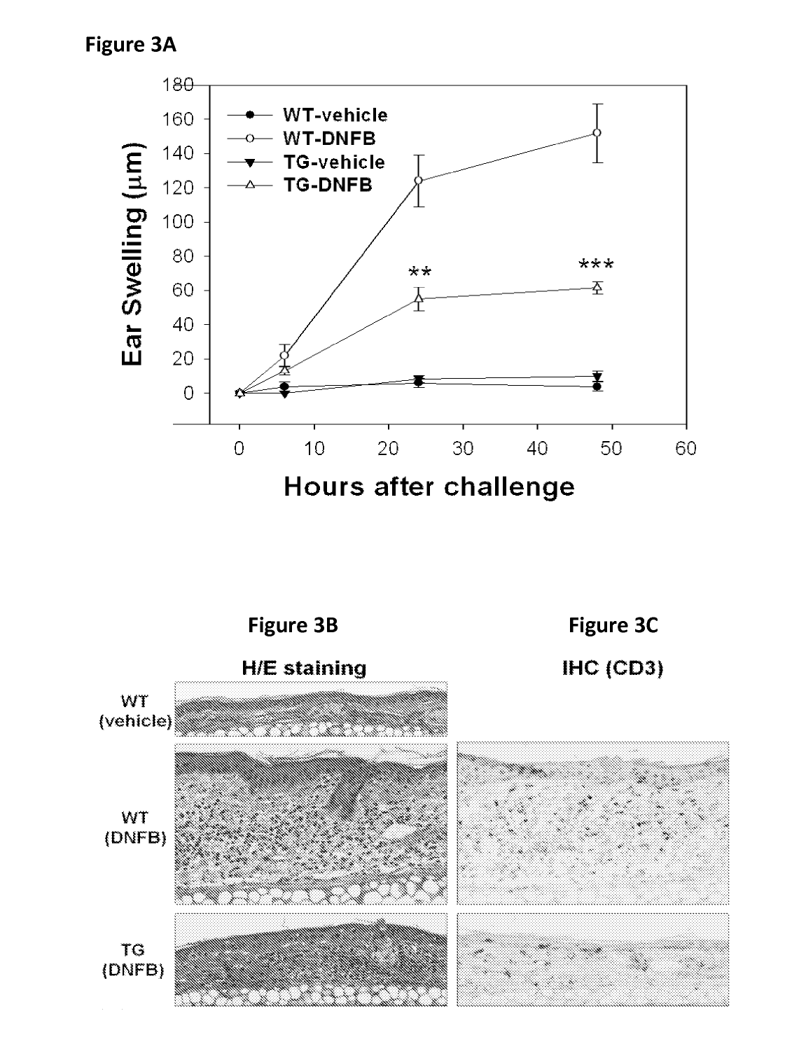

IL-37 is a homolog of the IL-1 cytokine family that is detected in lymph nodes, thymus, bone marrow and placenta. The IL-37 protein remains in the intracellular compartment because it contains no signal peptide. IL-37 expression is induced in response to pro-inflammatory stimuli and overexpression of IL-37 in monocytic or epithelial cells results in near complete suppression of pro-inflammatory cytokines. Transgenic mice expressing IL-37 are protected from non-lethal LPS-induced septic shock. The present inventors have also demonstrated that hapten-induced contact hypersensitivity, a classic model of adaptive cutaneous immune response, is suppressed by IL-37 (FIG. 3 and Example 3 infra). Additionally, IL-37 is induced in human blood cells after cultivation with the supernatant from human cancer cells (FIG. 3). Furthermore, murine cancer cells, B16F10, grow faster in IL-37 transgenic mice. These data suggest that soluble mediators derived from human cancer cells induce immunosuppression by upregulating anti-inflammatory IL-37 expression in blood cells, leading to enhanced tumorigenesis and progression (FIG. 1, B). These data suggest that IL-37 is a natural inhibitor of innate and adaptive immune response, and thus, inhibition of IL-37 may be an effective anti-inflammatory and/or cancer treatment.

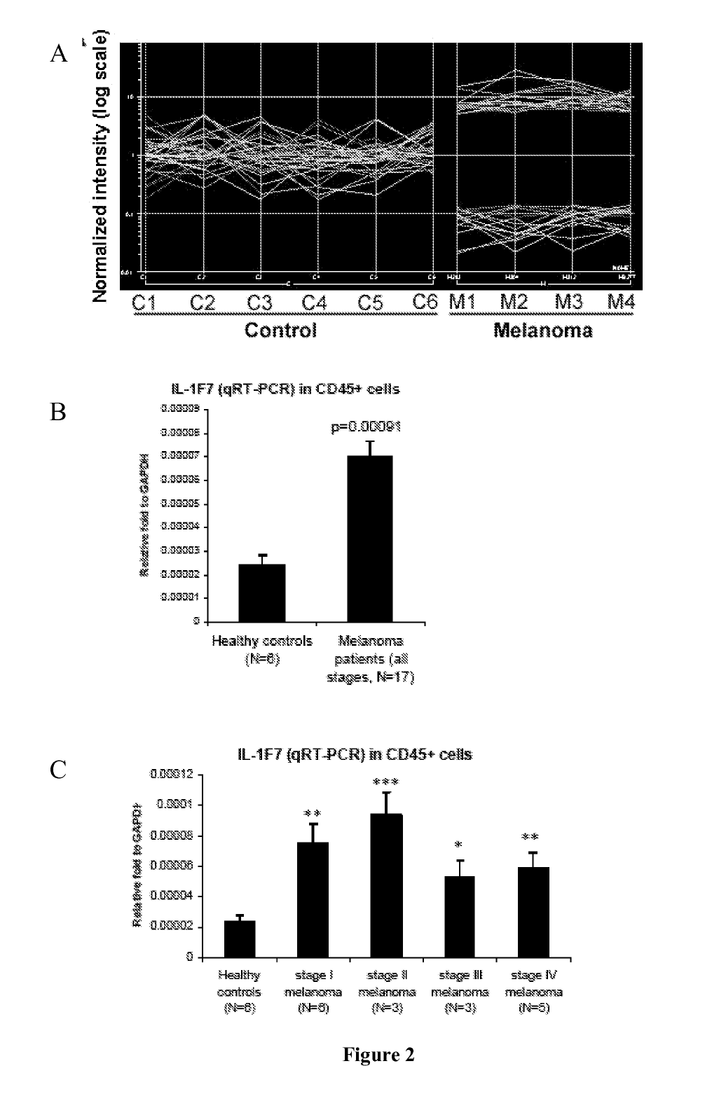

The level of the IL-37 marker of the present invention can be evaluated in blood samples obtained from cancer patients and compared with the levels measured in non-tumor samples obtained from the same patients. Measurement values of the biomarker were found to differ in biological samples from patients with melanoma as compared to biological samples from normal controls and these differences were statistically significant (FIG. 2 and Example 1 infra; Table 1 and Example 4 infra). Accordingly, the IL-37 biomarker is an indicator of cancer and the present invention includes all methods relying on correlations between the biomarker described herein and the presence of cancer, early stage and late stage cancer.

In one embodiment, the invention provides methods for determining whether a candidate drug is effective at treating cancer by evaluating the effect it has on the IL-37 biomarker values. In this context, the term “effective” is to be understood broadly to include reducing or alleviating the signs or symptoms of cancer, improving the clinical course of the disease, or reducing in any other objective or subjective indicia of the disease. Different drugs, doses and delivery routes can be evaluated by performing the method using different drug administration conditions. The method may also be used to compare the efficacy of two different drugs or other treatments or therapies for cancer.

It is expected that the IL-37 biomarker described herein will be measured in combination with other signs, symptoms and clinical tests of cancer, such as skin examination, dermoscopy, lymph node examination, chest x-ray, CT scan of the chest, head, abdomen, or pelvis, magnetic resonance imaging (MRI), and/or serum lactate dehydrogenase blood tests. Measurement of the biomarker of the invention along with any other marker known in the art, including those not specifically listed herein, falls within the scope of the present invention. Additionally, in preferred embodiments, the IL-37 biomarker described herein will be measured in conjunction with clinical testing of the subject to rule out concurrent infection in the subject, and/or clinical testing to rule out the presence of an autoimmune disorder in the subject.

The practice of the invention employs, unless otherwise indicated, conventional methods of analytical biochemistry, microbiology, molecular biology and recombinant DNA generally known techniques within the skill of the art. Such techniques are explained fully in the literature. (See, e.g., Sambrook et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (Glover, ed.); Oligonucleotide Synthesis (Gait, ed., Current Edition); Nucleic Acid Hybridization (Hames & Higgins, eds., Current Edition); Transcription and Translation (Hames & Higgins, eds., Current Edition); CRC Handbook of Parvoviruses, Vol. I & II (Tijessen, ed.); Fundamental Virology, 2nd Edition, Vol. I & II (Fields and Knipe, eds.)).

As used herein, a component (e.g., a marker) is referred to as “differentially expressed” in one sample as compared to another sample when the method used for detecting the component provides a different level or activity when applied to the two samples. A component is referred to as “increased” or “upregulated” in the first sample if the method for detecting the component indicates that the level or activity of the component is higher in the first sample than in the second sample (or if the component is detectable in the first sample but not in the second sample). Conversely, a component is referred to as “decreased” or “downregulated” in the first sample if the method for detecting the component indicates that the level or activity of the component is lower in the first sample than in the second sample (or if the component is detectable in the second sample but not in the first sample). In particular, marker is referred to as “increased” (“upregulated”) or “decreased” (“downregulated”) in a sample (or set of samples) obtained from a cancer subject (or a subject who is suspected of having cancer, or is at risk of developing cancer) if the level or activity of the marker is higher or lower, respectively, compared to the level of the marker in a sample (or set of samples) obtained from a non-cancer subject, or a reference value or range.

Comparative cDNA microarray analysis of whole blood samples from newly diagnosed cancer patients (stage IV, with no treatment) and from normal subjects was used to identify candidate genes from human cancer patients. Accordingly, in one aspect, the invention provides a biomarker of cancer. In another embodiment, the invention provides an isolated IL-37 biomarker or fragment thereof. As used herein, a compound is referred to as “isolated” when it has been separated from at least one component with which it is naturally associated. For example, a metabolite can be considered isolated if it is separated from contaminants including polypeptides, polynucleotides and other metabolites. Isolated molecules can be either prepared synthetically or purified from their natural environment. Standard quantification methodologies known in the art can be employed to obtain and isolate the molecules of the invention.

In particular, the inventors have discovered a unique biomarker associated with cancer, including early stage cancer, and cancer treatment outcome. The biomarker expression signature discriminates cancer tumors from non-tumorous tissue.

In one embodiment, the invention provides a marker of cancer in which the marker is the IL-37 biomarker, a fragment, precursor, successor, or modified version thereof.

In one embodiment, the invention provides a marker of early stage cancer, defined as stage IA or stage IB, in which the marker is an IL-37 protein or a fragment, precursor, successor, or modified version of the IL-37 protein or a molecule that comprises the foregoing fragment, precursor, successor or modified polypeptide.

In another embodiment, the invention provides a marker of early stage cancer, defined as stage IA or stage IB, in which the marker is an IL-37 gene, RNA transcript, or cDNA transcript or a fragment, precursor, successor, or modified version of the IL-37 gene, or a molecule that comprises the foregoing fragment, precursor, successor or modified gene.

In one embodiment, the IL-37 biomarker is associated with cancer patient prognosis.

In one embodiment, the IL-37 biomarker is associated with an aggressive form of cancer (i.e. stage III or IV disease).

In one embodiment, the cancer is a leukemia, pancreatic cancer, breast cancer, ovarian cancer, prostate cancer, lung cancer, or skin cancer. In a preferred embodiment, the cancer is melanoma.

Some variation is inherent in the measurements of the physical and chemical characteristics of the IL-37 biomarker. The magnitude of the variation depends to some extent on the reproducibility of the separation means and the specificity and sensitivity of the detection means used to make the measurement. Preferably, the method and technique used to measure the marker is sensitive and reproducible.

In one embodiment, the expression of IL-37 genes is detected by measuring amounts of transcripts of the IL-37 gene in cells in a biological sample. The expression of the genes may be detected by detecting hybridization of at least a portion of the IL-37 gene or a transcript thereof, to a nucleic acid molecule comprising a portion of the gene and a transcript thereof in a nucleic acid array. In another aspect the expression of the gene is detected by detecting the production of proteins encoded by the genes.

Another embodiment of the present invention relates to an assay system including a plurality of antibodies, or antigen binding fragments thereof, or aptamers for the detection of the differential expression of the IL-37 biomarker in patients with cancer. The plurality of antibodies, or antigen binding fragments thereof, or aptamers consists of antibodies, or antigen binding fragments thereof, or aptamers that selectively bind to IL-37 proteins differentially expressed in patients with cancer, and that can be detected as protein products using antibodies or aptamers. In addition, the plurality of antibodies, or antigen binding fragments thereof, or aptamers comprise antibodies, or antigen binding fragments thereof, or aptamers that selectively bind to IL-37 proteins or portions thereof (e.g., peptides) encoded by IL-37 genes.

Certain embodiments of the present invention utilize a plurality of biomarkers, including the IL-37 biomarker identified herein as being differentially expressed in subjects with cancer. As used herein, the terms “patient,” “subject” and “a subject who has cancer” and “cancer subject” are intended to refer to subjects who have been diagnosed with cancer. The terms “non-subject” and “a subject who does not have cancer” are intended to refer to a subject who has not been diagnosed with cancer, or who is cancer-free as a result of surgery to remove the diseased tissue. A non-cancer subject may be healthy and have no other disease, or they may have a disease other than cancer.

The IL-37 biomarker of the invention is useful in methods for diagnosing cancer, determining the extent and/or severity of the disease, monitoring progression of the disease and/or response to therapy. Such methods can be performed in human and non-human subjects. The IL-37 biomarker is also useful in methods for treating cancer and for evaluating the efficacy of treatment for the disease. Such methods can be performed in human and non-human subjects. The marker may also be used as pharmaceutical compositions or in kits. The marker may also be used to screen candidate compounds that modulate the expression of the IL-37 biomarker. The marker may also be used to screen candidate drugs for treatment of cancer. Such screening methods can be performed in human and non-human subjects.

The IL-37 biomarker may be isolated by any suitable method known in the art. The IL-37 biomarker can be purified from natural sources by standard methods known in the art (e.g., chromatography, centrifugation, differential solubility, immunoassay). In one embodiment, the IL-37 biomarker may be isolated from a biological sample using the methods disclosed herein. In another embodiment, polypeptide marker may be isolated from a sample by contacting the sample with substrate-bound antibodies or aptamers that specifically bind to the marker.

The present invention also encompasses molecules which specifically bind the IL-37 biomarker. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁴ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The binding molecules include antibodies, aptamers and antibody fragments. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab') fragments, fusion proteins and any modifications of the foregoing that comprise an antigen recognition site of the required specificity. As used herein, an aptamer is a non-naturally occurring nucleic acid having a desirable action on a target, including, but not limited to, binding of the target, catalytically changing the target, reacting with the target in a way which modifies/alters the target or the functional activity of the target, covalently attaching to the target as in a suicide inhibitor, facilitating the reaction between the target and another molecule. In a preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

In one embodiment, the antibodies or aptamers specifically bind to a component that is a fragment, modification, precursor or successor of an IL-37 biomarker.

Another embodiment of the present invention relates to a plurality of aptamers, antibodies, or antigen binding fragments thereof, for the detection of the expression of the IL-37 biomarker differentially expressed in patients with cancer. The plurality of aptamers, antibodies, or antigen binding fragments thereof, consists of antibodies, or antigen binding fragments thereof, that selectively bind to IL-37 proteins differentially expressed in patients with cancer, and that can be detected using antibodies or aptamers. In addition, the plurality of aptamers, antibodies, or antigen binding fragments thereof, comprises antibodies, or antigen binding fragments thereof, that selectively bind to proteins or portions thereof (peptides) encoded by any of the genes from the tables provided herein.

According to the present invention, a plurality of aptamers, antibodies, or antigen binding fragments thereof, refers to at least 2, at least 3, at least 4, at least 5, at least 6, at least 7, at least 8, at least 9, at least 10, and so on, in increments of one, up to any suitable number of antibodies, or antigen binding fragments thereof, including, in one embodiment, antibodies representing all of the biomarker described herein, or antigen binding fragments thereof.

Certain antibodies that specifically bind polypeptide and/or polynucleotide IL-37 biomarkers may already be known and/or available for purchase from commercial sources. In any event, the antibodies of the invention may be prepared by any suitable means known in the art. For example, antibodies may be prepared by immunizing an animal host with an IL-37 biomarker, or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies or aptamers that specifically bind the IL-37 biomarker can be used, for example, in methods for detecting cancer in a patient. In some embodiments the antibodies are conjugated to a detection molecule or moiety (e.g., a dye, and enzyme) and can be used in ELISA or sandwich assays to detect marker of the invention.

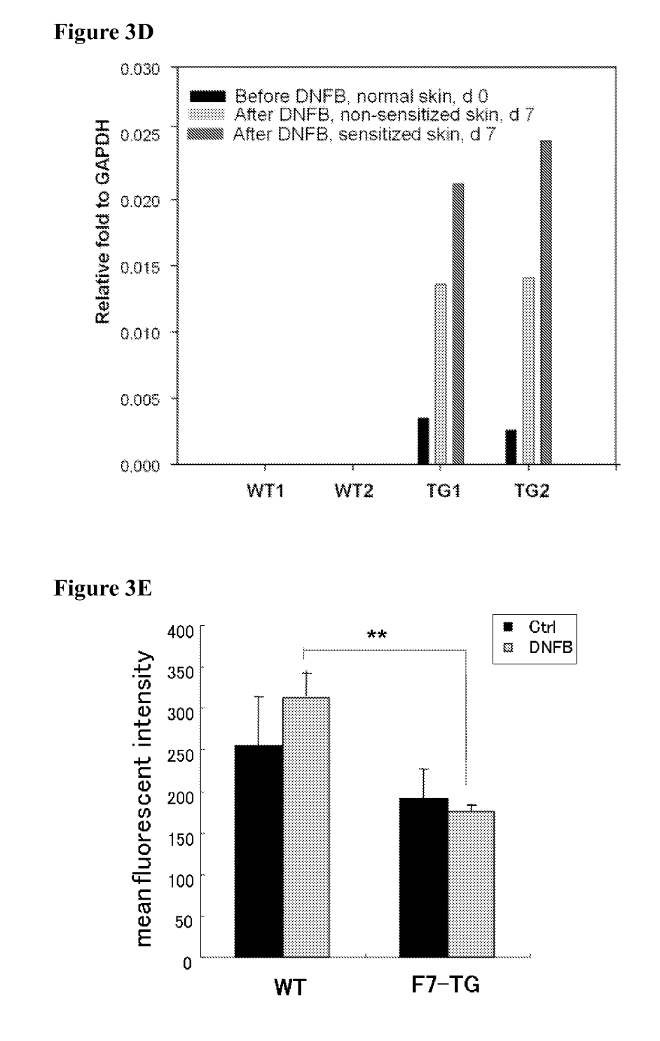

In another embodiment, antibodies or aptamers against a polypeptide or polynucleotide IL-37 biomarker can be used to assay a tissue sample (e.g., a hepatocellular tumor tissue) for the IL-37 biomarker. The antibodies or aptamers specifically bind to the IL-37 biomarker, if any, present in the tissue sections and allow the localization of the biomarker in the tissue (FIG. 4, infra). Similarly, antibodies or aptamers labeled with a radioisotope may be used for in vivo imaging or treatment applications.

Another aspect of the invention provides compositions comprising a marker of the invention, a binding molecule that is specific for a marker (e.g., an antibody or an aptamer), an inhibitor of a marker, or other molecule that can increase or decrease the level or activity of a polypeptide marker or polynucleotide marker. Such compositions may be pharmaceutical compositions formulated for use as a therapeutic.

Alternatively, the invention provides a composition that comprises a fragment, modification, precursor or successor of a IL-37 biomarker.

In another embodiment, the invention provides a composition that comprises an antibody or aptamer that specifically binds to a IL-37 polypeptide or a molecule that comprises such antibody or aptamer.

The present invention also provides methods of detecting the IL-37 biomarker. The practice of the present invention employs, unless otherwise indicated, conventional methods of analytical biochemistry, microbiology, molecular biology and recombinant DNA techniques within the skill of the art. Such techniques are explained fully in the literature. (See, e.g., Sambrook, J. et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

The marker of the invention may be detected by any method known to those of skill in the art, including without limitation LC-MS, GC-MS, immunoassays, microarray analysis, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a marker is included in the invention. It is within the ability of one of ordinary skill in the art to determine which method would be most appropriate for measuring a specific marker. Thus, for example, an ELISA assay may be best suited for use in a physician's office while a measurement requiring more sophisticated instrumentation may be best suited for use in a clinical laboratory. Regardless of the method selected, it is important that the measurements be reproducible.

The marker of the invention can be measured by mass spectrometry, which allows direct measurements of analytes with high sensitivity and reproducibility. A number of mass spectrometric methods are available. As will be appreciated by one of skill in the art, many separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

In other embodiments, the level of the IL-37 biomarker may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker. Standard protocols and data analysis are used to determine the marker concentrations from the assay data.

A number of the assays discussed above employ a reagent that specifically binds to the marker. Any molecule that is capable of specifically binding to an IL-37 biomarker is included within the invention. In some embodiments, the binding molecules are antibodies or antibody fragments. In other embodiments, the binding molecules are non-antibody species, such as aptamers. Thus, for example, the binding molecule may be an enzyme for which the marker is a substrate. The binding molecules may recognize any epitope of the targeted marker.

As described above, the binding molecules may be identified and produced by any method accepted in the art. Methods for identifying and producing antibodies and antibody fragments specific for an analyte are well known. Examples of other methods used to identify the binding molecules include binding assays with random peptide libraries (e.g., phage display) and design methods based on an analysis of the structure of the IL-37 biomarker.

The IL-37 biomarker of the invention also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

In one aspect, the present invention provides a method for determining whether a subject has cancer (i.e. diagnosing cancer in a subject). These methods comprise obtaining a biological sample from a subject suspected of having cancer, or at risk for developing cancer, detecting the level or activity of one or more biomarker in the sample, and comparing the result to the level or activity of the IL-37 biomarker in a sample obtained from a non-cancer subject, or to a reference range or value.

As used herein, the term “biological sample” includes a sample from any body fluid or tissue (e.g., blood, cultured whole blood, cerebrospinal fluid, urine, saliva, cancer tissue, total RNA isolated from blood, peritoneal fluids, plural fluids, tears, sweat). Typically, the standard IL-37 biomarker level or reference range is obtained by measuring the IL-37 biomarker in a set of normal controls. Measurement of the standard IL-37 biomarker level or reference range need not be made contemporaneously; it may be a historical measurement. Preferably, the normal control is matched to the patient with respect to some attribute(s) (e.g., age). Depending upon the difference between the measured and standard level or reference range, the patient can be diagnosed as having cancer or as not having cancer. In some embodiments, cancer is diagnosed in the patient if the expression level of the IL-37 biomarker in the patient sample is statistically more similar to the expression level of the IL-37 biomarker that has been associated with cancer than the expression level of the IL-37 biomarker that has been associated with the not tnal controls.

Any and all of the various forms of cancer are intended to be within the scope of the present invention. Indeed, by providing a method for categorizing patients based on IL-37 biomarker measurement level, the compositions and methods of the present invention may be used to uncover and define various forms of the disease.

The methods of the present invention may be used to make the diagnosis of cancer, independently from other information such as the patient's symptoms or the results of other clinical or paraclinical tests. However, the methods of the present invention may be used in conjunction with such other data points.

Because a diagnosis is rarely based exclusively on the results of a single test, the methods of the invention may be used to determine whether a subject is more likely than not to have cancer, or is more likely to have cancer than to have another disease, based on the difference between the measured and standard level or reference range of the IL-37 biomarker. Thus, for example, a patient with a putative diagnosis of cancer may be diagnosed as being “more likely” or “less likely” to have cancer in light of the information provided by a method of the present invention. If a plurality of biomarkers are measured, at least one and up to all of the measured biomarkers, including IL-37, must differ, in the appropriate direction, for the subject to be diagnosed as having (or being more likely to have) cancer.

The biological sample may be of any tissue or fluid, including a serum or tissue sample, but other biological fluids or tissue may be used. Possible biological fluids include, but are not limited to, blood, cultured whole blood, urine, saliva, and skin tissue. In some embodiments, the level of the IL-37 biomarker may be compared to the level of another marker or some other component in a different tissue, fluid or biological compartment. Thus, a differential comparison may be made of an IL-37 biomarker in tissue and serum. It is also within the scope of the invention to compare the level of an IL-37 biomarker with the level of another biomarker or some other component within the same compartment.

As will be apparent to those of ordinary skill in the art, the above description is not limited to making an initial diagnosis of cancer, but also is applicable to confirming a provisional diagnosis of cancer or “ruling out” such a diagnosis. Furthermore, an increased level or activity of the IL-37 biomarker in a sample obtained from a subject suspected of having cancer, or at risk for developing cancer, is indicative that the subject has or is at risk for developing cancer. In a specific embodiment, an increased expression level of the IL-37 biomarker in a sample obtained from a subject suspected of having cancer, or at risk for developing cancer, is indicative that the subject has or is at eleveated risk for developing cancer. In another specific embodiment, an increased expression level of the IL-37 biomarker in a sample obtained from a subject suspected of having cancer is indicative that the subject has cancer. In another specific embodiment, an increased expression level of the IL-37 biomarker in a sample obtained from a subject suspected of having cancer is indicative that the subject has a melanoma.

The invention also provides a method for determining a subject's risk of developing cancer, the method comprising obtaining a biological sample from a subject, detecting the level or activity of the IL-37 biomarker in the sample, and comparing the result to the level or activity of the biomarker in a sample obtained from a non-cancer subject, or to a reference range or value wherein an increase or decrease of the IL-37 biomarker is correlated with the risk of developing cancer.

The present inventors have found that expression levels of IL-37 in melanoma patients were nearly 5-fold greater than IL-37 expression levels in healthy controls (Example 4, Table 1 infra). Therefore, in these embodiments of the invention, the increased expression and/or activity level of IL-37, indicative of cancer in a subject, or a subject's risk of developing cancer, may be an increase of at least two-fold (2×), three-fold (3×), four-fold (4×), five-fold (5×), or greater than five-fold (>5×) over the expression and/or activity from a healthy control.

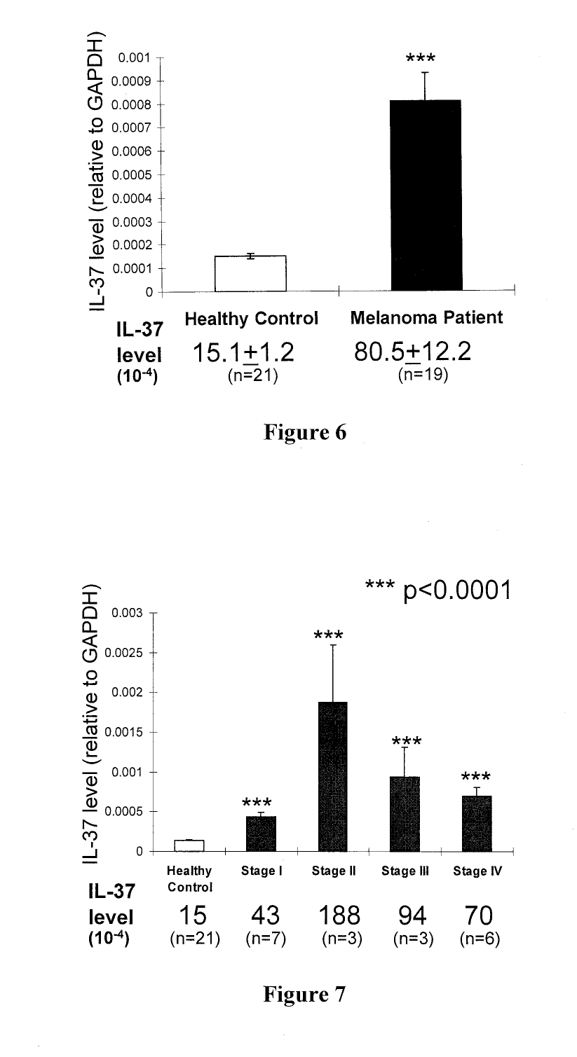

The invention also provides methods for determining the stage or severity of cancer, the method comprising obtaining a biological sample from a subject, detecting the level or activity of a marker in the sample, and comparing the result to the level or activity of the marker in a sample obtained from a non-cancer subject, or to a reference range or value wherein an increase or decrease of the marker is correlated with the stage or severity of the disease. The present inventors have found that expression levels of IL-37 in melanoma patients were significantly different than IL-37 expression levels in healthy controls (Example 4, FIG. 7 infra).

Thus, in a specific embodiment, an increased expression level of the IL-37 biomarker in a sample obtained from a subject suspected of having cancer (relative to the expression level of the IL-37 biomarker in a sample obtained from a healthy control) is indicative that the subject has a Stage I melanoma. In a specific embodiment, an expression level of the IL-37 biomarker that is at least 2-fold greater in a sample obtained from a subject suspected of having cancer is indicative that the subject has a Stage I melanoma. In another specific embodiment, an expression level of the IL-37 biomarker that is at least 3-fold greater in a sample obtained from a subject suspected of having cancer is indicative that the subject has a Stage I melanoma.

In a specific embodiment, an increased expression level of the IL-37 biomarker in a sample obtained from a subject suspected of having cancer (relative to the expression level of the IL-37 biomarker in a sample obtained from a healthy control) is indicative that the subject has a Stage II melanoma. In a specific embodiment, an expression level of the IL-37 biomarker that is at least 5-fold greater in a sample obtained from a subject suspected of having cancer is indicative that the subject has a Stage II melanoma. In another specific embodiment, an expression level of the IL-37 biomarker that is at least 8-fold greater in a sample obtained from a subject suspected of having cancer is indicative that the subject has a Stage II melanoma. In another specific embodiment, an expression level of the IL-37 biomarker that is at least 10-fold greater in a sample obtained from a subject suspected of having cancer is indicative that the subject has a Stage II melanoma. In another specific embodiment, an expression level of the IL-37 biomarker that is at least 12-fold greater in a sample obtained from a subject suspected of having cancer is indicative that the subject has a Stage II melanoma.

In a specific embodiment, an increased expression level of the IL-37 biomarker in a sample obtained from a subject suspected of having cancer (relative to the expression level of the IL-37 biomarker in a sample obtained from a healthy control) is indicative that the subject has a Stage III melanoma. In a specific embodiment, an expression level of the IL-37 biomarker that is at least 4-fold greater in a sample obtained from a subject suspected of having cancer is indicative that the subject has a Stage III melanoma. In another specific embodiment, an expression level of the IL-37 biomarker that is at least 5-fold greater in a sample obtained from a subject suspected of having cancer is indicative that the subject has a Stage III melanoma. In another specific embodiment, an expression level of the IL-37 biomarker that is at least 6-fold greater in a sample obtained from a subject suspected of having cancer is indicative that the subject has a Stage III melanoma.

In a specific embodiment, an increased expression level of the IL-37 biomarker in a sample obtained from a subject suspected of having cancer (relative to the expression level of the IL-37 biomarker in a sample obtained from a healthy control) is indicative that the subject has a Stage IV melanoma. In a specific embodiment, an expression level of the IL-37 biomarker that is at least 2-fold greater in a sample obtained from a subject suspected of having cancer is indicative that the subject has a Stage IV melanoma. In another specific embodiment, an expression level of the IL-37 biomarker that is at least 3-fold greater in a sample obtained from a subject suspected of having cancer is indicative that the subject has a Stage IV melanoma. In another specific embodiment, an expression level of the IL-37 biomarker that is at least 4-fold greater in a sample obtained from a subject suspected of having cancer is indicative that the subject has a Stage IV melanoma.

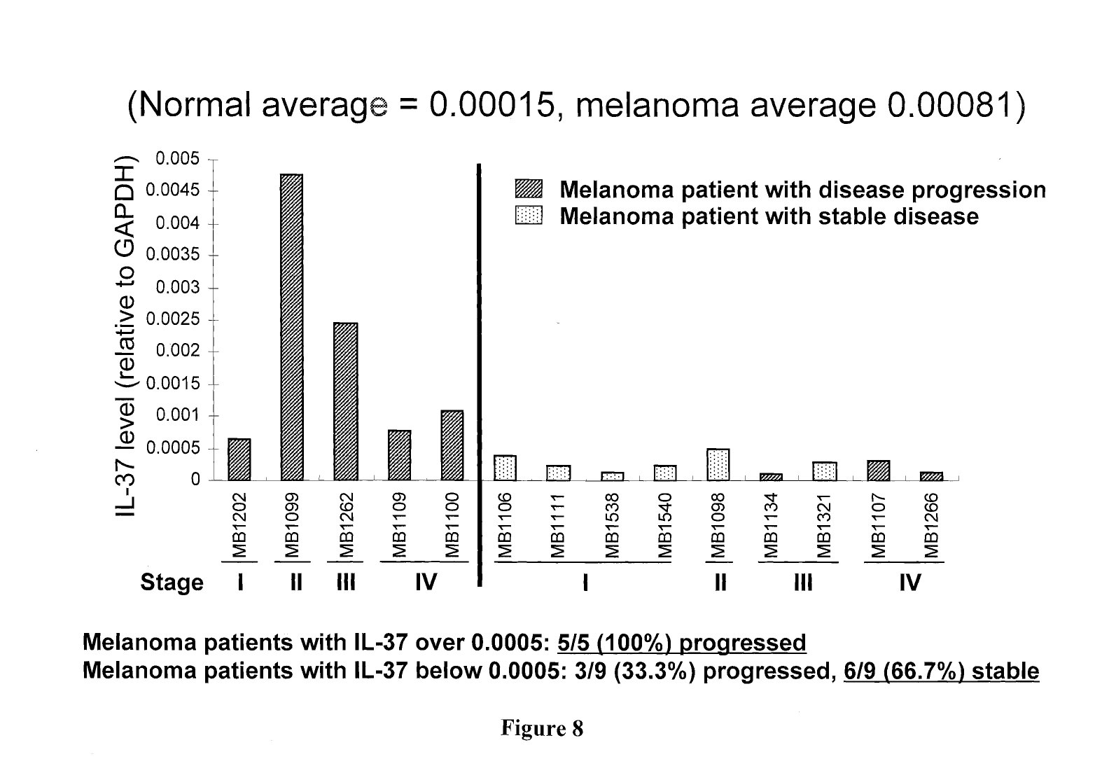

In another aspect, the invention provides methods for monitoring the progression of the disease in a subject who has cancer, the method comprising obtaining a first biological sample from a subject, detecting the level or activity of a IL-37 biomarker in the sample, and comparing the result to the level or activity of the marker in a second sample obtained from the subject at a later time, or to a reference range or value wherein an increase or decrease of the IL-37 biomarker is correlated with progression of the disease. The present inventors have discovered that elevated IL-37 expression levels are indicative of disease progression (for example, stage I/II to stage III or IV; stage III to stage IV; stage IV to death) in a cancer patient (Example 5, FIG. 8 infra), whereas low IL-37 expression levels are indicative of stable disease in a cancer patient.

Thus, in a specific embodiment, an increased expression level of the IL-37 biomarker in a sample obtained from a subject diagnosed with a cancer (relative to the expression level of the IL-37 biomarker in a sample obtained from a healthy control) is indicative that the subject has a melanoma and the cancer is progressing. In a specific embodiment, an expression level of the IL-37 biomarker that is at least 3-fold greater in a sample obtained from a subject with a progressing cancer. In another specific embodiment, an expression level of the IL-37 biomarker that is at least 4-fold greater in a sample obtained from a subject with a progressing cancer.

Thus, in another specific embodiment, a moderately-increased expression level of the IL-37 biomarker (relative to the expression level of the IL-37 biomarker in a sample obtained from a healthy control) in a sample obtained from a subject diagnosed with a cancer is indicative that the subject has a melanoma and the disease is stable, i.e. is not progressing. In a specific embodiment, the expression level of the IL-37 biomarker is about 2-fold greater in a sample obtained from a subject with a stable cancer than the expression level of the IL-37 biomarker obtained from a sample from a healthy control subject. In a specific embodiment, the expression level of the IL-37 biomarker is less than 2-fold greater in a sample obtained from a subject with a stable cancer than the expression level of the IL-37 biomarker obtained from a sample from a healthy control subject. In a specific embodiment, the expression level of the IL-37 biomarker is between about 2-fold and about 3-fold greater in a sample obtained from a subject with a stable cancer than the expression level of the IL-37 biomarker obtained from a sample from a healthy control subject.

Cancer prognosis generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

A significant difference in the elevation of the measured IL-37 biomarker value indicates that the patient has (or is more likely to have, or is at risk of having, or is at risk of developing, and so forth) cancer. Measurements can be of (i) an IL-37 biomarker of the present invention, or an IL-37 biomarker of the present invention and another factor known to be associated with cancer (e.g., serum lactate dehydrogenase). Furthermore, the amount of change in a biomarker level may be an indication of the relative likelihood of the presence of cancer.

The IL-37 biomarker may be detected in any biological sample obtained from the subject, by any suitable method known in the art (e.g., immunoassays, hybridization assay) see supra. In some embodiments, the IL-37 biomarker is detected in a tumor sample obtained from the patient by surgical procedure(s).

In an alternative embodiment of the invention, a method is provided for monitoring a cancer patient over time to determine whether the disease is progressing. The specific techniques used in implementing this embodiment are similar to those used in the embodiments described above. The method is performed by obtaining a biological sample, such as serum or tissue, from the subject at a certain time (t₁); measuring the level of the IL-37 biomarker in the biological sample; and comparing the measured level with the level measured with respect to a biological sample obtained from the subject at an earlier time (t₀). Depending upon the difference between the measured levels, it can be seen whether the IL-37 biomarker level has increased, decreased, or remained constant over the interval (t₁-t₀). A further elevation of the IL-37 biomarker would suggest a progression of the disease during the interval. Subsequent sample acquisitions and measurements can be performed as many times as desired over a range of times t₂ to tₙ.

The ability to monitor a patient by making serial IL-37 biomarker level determinations represents a valuable clinical tool. Rather than the limited “snapshot” provided by a single test, such monitoring reveals trends in IL-37 biomarker levels over time. In addition to indicating a progression of the disease, tracking the IL-37 biomarker levels in a patient could be used to predict exacerbations or indicate the clinical course of the disease. For example, as will be apparent to one of skill in the art, the IL-37 biomarker could be further investigated to distinguish between any or all of the known forms of cancer or any later described types or subtypes of the disease. In addition, the sensitivity and specificity of any method of the present invention could be further investigated with respect to distinguishing cancer from other diseases or to predict relapse or remission.

In an analogous manner, administration of a chemotherapeutic drug or drug combination can be evaluated or re-evaluated in light of the assay results of the present invention. For example, the drug(s) can be administered differently to different subject populations, and measurements corresponding to administration analyzed to determine if the differences in the IL-37 biomarker level before and after drug administration are significant. Results from the different drug regiments can also be compared with each other directly. Alternatively, the assay results may indicate the desirability of one drug regimen over another, or indicate that a specific drug regimen should or should not be administered to a cancer patient. In one embodiment, the finding of elevated levels of the IL-37 biomarker of the present invention in a cancer patient is indicative of a good prognosis for response to treatment with chemotherapeutic agents. In another embodiment, the absence of elevated levels of the IL-37 biomarker of the present invention in a cancer patient is indicative of a poor prognosis for response to treatment.

In another aspect, the invention provides methods for screening candidate compounds for use as therapeutic compounds. In one embodiment, the method comprises screening candidate compounds for those that provide clinical progress following administration to a cancer patient from which a tumor sample has been shown to have elevated levels of the IL-37 biomarker.

In an analogous manner, the IL-37 biomarker can be used to assess the efficacy of a therapeutic intervention in a subject. The same approach described above would be used, except a suitable treatment would be started, or an ongoing treatment would be changed, before the second measurement (i.e., after t₀ and before t₁). The treatment can be any therapeutic intervention, such as drug administration, dietary restriction or surgery, and can follow any suitable schedule over any time period as appropriate for the intervention. The measurements before and after could then be compared to determine whether or not the treatment had a desired therapeutic effect. As will be appreciated by one of skill in the art, the determination may be confounded by other superimposed processes (e.g., an exacerbation of the disease during the same period).

In a further embodiment, the IL-37 biomarker may be used to screen candidate drugs, for example, in a clinical trial, to determine whether a candidate drug is effective in treating cancer. At time t₀, a biological sample is obtained from each subject in population of subjects diagnosed with cancer. Next, assays are performed on each subject's sample to measure levels of a biological marker. In some embodiments, only the IL-37 biomarker is monitored, while in other embodiments, a combination of markers, including a IL-37 biomarker, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. At time t₁, after drug administration, a biological sample is acquired from the sub-population and the same assays are performed on the biological samples as were previously performed to obtain IL-37 biomarker measurement values. As before, subsequent sample acquisitions and measurements can be performed as many times as desired over a range of times t₂ to t₀. In such a study, a different sub-population of the subject population serves as a control group, to which a placebo is administered. The same procedure is then followed for the control group: obtaining the biological sample, processing the sample, and measuring the biological marker to obtain a IL-37 biomarker measurement chart.

Specific doses and delivery routes can also be examined. The method is performed by administering the candidate drug at specified dose or delivery routes to subjects with cancer; obtaining biological samples, such as serum or tissue, from the subjects; measuring the level of at least one of the biomarker in each of the biological samples; and, comparing the measured level for each sample with other samples and/or a standard level. Typically, the standard level is obtained by measuring the IL-37 biomarker in the subject before drug administration. Depending upon the difference between the measured and standard levels, the drug can be considered to have an effect on cancer. If multiple biomarker levels are measured, at least one and up to all of the biomarkers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating cancer.

In a typical embodiment, a subject population having cancer is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed. The stratification of the patient population may be made based on the levels of the IL-37 biomarker.

In general, a number of statistical considerations must be made in designing the trial to ensure that statistically significant changes in biomarker measurements can be detected following drug administration. The amount of change in a biomarker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time t₀, a biological sample is obtained from each subject in the population. Next, an assay or variety of assays is performed on each subject's sample to measure levels of particular biomarker of the invention. The assays can use conventional methods and reagents, as described above. If the sample is blood, then the assays typically are performed on either serum or plasma. For other fluids or tissues, additional sample preparation steps are included as necessary before the assays are performed. The assays measure values of at least one of the biological marker described herein. In some embodiments, only the IL-37 biomarker is monitored, while in other embodiments, a combination of factors are monitored. The IL-37 biomarker may also be monitored in conjunction with other measurements and factors associated with cancer (e.g., MRI imaging). The number of biological markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner as the sample acquired from the subject population before drug administration (the “t₀ sample”). The same assays are performed on the t₁ sample as on the t₀ sample to obtain measurement values. Subsequent sample acquisitions and measurements can be performed as many times as desired over a range of times t₂ to t₀.

Typically, a different sub-population of the subject population is used as a control group, to which a placebo is administered. The same procedure is then followed for the control group: obtaining the biological sample, processing the sample, and measuring the biological marker to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the biomarker(s), including the IL-37 biomarker, are now available for each subject. The different measurement values are compared and analyzed to determine whether the biological marker changed in the expected direction for the drug group but not for the placebo group, indicating that the candidate drug is effective in treating the disease. In preferred embodiments, such change is statistically significant. The measurement values at time t₁ for the group that received the candidate drug are compared with standard measurement values, preferably the measured values before the drug was given to the group, i.e., at time t₀. Typically, the comparison takes the form of statistical analysis of the measured values of the entire population before and after administration of the drug or placebo. Any conventional statistical method can be used to determine whether the changes in biological marker values are statistically significant. For example, paired comparisons can be made for each biomarker using either a parametric paired t-test or a non-parametric sign or sign rank test, depending upon the distribution of the data.

In addition, tests may be performed to ensure that statistically significant changes found in the drug group are not also found in the placebo group. Without such tests, it cannot be determined whether the observed changes occur in all patients and are therefore not a result of candidate drug administration.

A significant decrease the IL-37 biomarker levels may indicate that the drug is effective. If more than one biomarker is measured, then drug efficacy can be indicated by change in only one biomarker, all biomarkers, or any number in between. Measurements can be of both the IL-37 biomarker and other measurements and factors associated with cancer (e.g., measurement of serum lactate dehydrogenase and/or CT imaging). Furthermore, the amount of change in the IL-37 biomarker level may be an indication of the efficacy of the drug.

In addition to determining whether a particular drug is effective in treating cancer, the IL-37 biomarker can also be used to examine dose effects of a candidate drug. There are a number of different ways that varying doses can be examined. For example, different doses of a drug can be administered to different subject populations, and measurements corresponding to each dose analyzed to determine if the differences in the IL-37 biomarker before and after drug administration are significant. In this way, a minimal dose required to effect a change can be estimated. In addition, results from different doses can be compared with each other to determine how each biomarker behaves as a function of dose. Based on the results of drug screenings, the IL-37 biomarker may be used as a theragnostic; that is, it may be used to individualize medical treatment.

In another aspect, the invention provides a kit for detecting the IL-37 biomarker. The kit may be prepared as an assay system including any one of assay reagents, assay controls, protocols, exemplary assay results, or combinations of these components designed to provide the user with means to evaluate the expression level of the IL-37 biomarker.

In another embodiment, the invention provides a kit for diagnosing cancer in a patient including reagents for detecting at least one polypeptide or polynucleotide IL-37 biomarker in a biological sample from a subject.

The kits of the invention may comprise one or more of the following: an antibody, wherein the antibody specifically binds with a marker, a labeled binding partner to the antibody, a solid phase upon which is immobilized the antibody or its binding partner, instructions on how to use the kit, and a label or insert indicating regulatory approval for diagnostic or therapeutic use.

The invention includes microarrays comprising the IL-37 biomarker, or molecules, such as antibodies, which specifically bind to the marker of the present invention. In this aspect of the invention, standard techniques of microarray technology are utilized to assess expression of the polypeptide the IL-37 biomarker and/or identify biological constituents that bind the IL-37 biomarker polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding. Arrays that bind the IL-37 biomarker of the invention also can be used for diagnostic applications, such as for identifying subjects that have a condition characterized by expression of polypeptide biomarker, e.g., cancer.

The assay system preferably also includes one or more controls. The controls may include: i) information containing a predetermined control level of IL-37 that has been correlated with cancer in a subject; ii) information containing a predetermined control level of IL-37 that has been correlated with melanoma in a subject; iii) information containing a predetermined control level of IL-37 that has been correlated with a stage of melanoma in a patient; iv) information containing a predetermined control level of IL-37 that has been correlated with cancer progression; v) information containing a predetermined control level of IL-37 that has been correlated with a stable cancer; and vi) combinations of these controls. The controls may include: (i) a control sample for detecting sensitivity to a chemotherapeutic agent or agents being evaluated for use in a patient; (ii) a control sample for detecting resistance to the chemotherapeutic(s); (iii) information containing a predetermined control level of the IL-37 biomarker to be measured with regard to the chemotherapeutic sensitivity or resistance (e.g., a predetermined control level of the IL-37 biomarker that has been correlated with sensitivity to the chemotherapeutic(s) or resistance to the chemotherapeutic).

In another embodiment, a means for detecting the expression level of the IL-37 biomarker can generally be any type of reagent that can include, but is not limited to, antibodies and antigen binding fragments thereof, peptides, binding partners, aptamers, enzymes, and small molecules. Additional reagents useful for performing an assay using such means for detection can also be included, such as reagents for performing immunohistochemistry or another binding assays.

The means for detecting of the assay system of the present invention can be conjugated to a detectable tag or detectable label. Such a tag can be any suitable tag which allows for detection of the reagents used to detect the marker of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., DynabeadsTM), fluorescent dyes (e.g., fluorescein, texas red, rhodamine, green fluorescent protein, and the like), radiolabels (e.g., 3H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay system of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

The assay system can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a biomarker described previously herein. Specifically, the means is characterized in that it identifies a specific marker of the cell type being analyzed that positively identifies the cell type. For example, in a cancer tumor assay, it is desirable to screen cancer cells for the level of the IL-37 biomarker expression and/or biological activity. Therefore, the means for detecting a control marker identifies a marker that is characteristic of a cancer cell, so that the cell is distinguished from other cell types, such as a connective tissue or inflammatory cells. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell marker are known in the art and can be used to produce such reagents for detection.

The assay systems and methods of the present invention can be used not only to identify patients that are predicted to survive or be responsive to treatment, but also to identify treatments that can improve the responsiveness of cancer cells which are resistant to treatment, and to develop adjuvant treatments that enhance the response of the treatment and survival.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Identification of Differentially-Expressed Pathways and Genes in Cancer Blood

cDNA microarray analysis was used to identify a panel of candidate genes from human cancer patients. Whole blood samples were obtained from 4 newly diagnosed cancer patients (stage IV, with no treatment) and 6 normal subjects. cDNA transcripts were hybridized to the Human Genome U133 Plus 2.0 array, according to Affymetrix instructions. 72 genes were selected based on the criteria using fold changes, expression levels and comparison replicates (FIG. 2A), and 39 genes were confirmed to be differentially expressed in cancer blood by qRT-PCR. Among these genes, a unique anti-inflammatory gene was identified, IL-37, which was highly expressed in the blood of stage IV cancer patients compared with that of controls, (FIG. 2B). qRT-PCR analysis of fractionated cells from cancer patients revealed that the gene was expressed in CD45+ (leukocytes) but not CD45− cells (such as circulating cancer cells, endothelial cells, fibroblasts, erythrocytes or platelets) even in stage I patients (FIG. 2C).

### Example 2

### IL-37 is Induced in Pro-Inflammatory Milieu

Steady-state constitutive and inducible IL-37 expression was assessed in peripheral blood mononuclear cells (PBMCs) from healthy donors. The TLR ligands (LPS and CpG) and pro-inflammatory cytokines IL-lb, TNFa, IL-18, IFNg and TGF-b1 increased synthesis, whereas IL-4 plus GM-CSF inhibited IL-37 expression.

IL-37 overexpression abrogates production of proinflammatory cytokines in RAW cells. Transfection of human IL-37b into a murine macrophage RAW cell line induced almost complete suppression of several major cytokines such as IL-la and IL-6. Levels of M-CSF, GM-CSF and IL-lb were also considerably reduced. On the other hand, MCP-1, IL-17, and IL-13 showed consistent increases.

LPS-induced endotoxic shock (innate immune response) is inhibited in IL-37 transgenic mice. In order to assess IL-37 in vivo, transgenic mice expressing human IL-37 (as the mouse counterpart of IL-37 has not yet been identified) were generated. Transgenic mice were protected from non-lethal LPS-induced endotoxic shock, exhibiting markedly improved lung and kidney function and reduced liver damage. Levels of circulating and tissue cytokines were reduced by 72-95% compared to wild-type (WT) mice, indicating that IL-37 is a natural inhibitor of innate immune responses.

### Example 3

### Contact Hypersensitivity to Dinitrofluorobenzene (DNFB) (Adaptive Immune Response) is Inhibited in IL-37 Transgenic Mice

The well-established model of contact hypersensitivity using the sensitizer DNFB was employed. Whereas wild type mice showed increased ear swelling at 24 and 48 hours after challenge (FIG. 3A, ◯), the response was reduced clinically and histologically in IL-37 TG mice (FIG. 3A, Δ and 3B). T cell infiltrates were reduced in the dermis of transgenic mouse ears after DNFB challenge (FIG. 3C), indicating that T-cell-mediated adaptive immune response is inhibited by IL-37. IL-37 protein expression was detected in dermal infiltrating cells with dendritic shape. IL-37 gene expression was low in the skin before DNFB stimulation (FIG. 3D, left black bars), but after 7 days, it was increased 10-fold in the local sensitized skin (FIG. 3D, right dark grey bars). Surprisingly, non-sensitized skin distant from the DNFB site exhibited a 7-fold increase on Day 7 compared to its baseline (FIG. 3D, middle bars) suggesting that IL-37 expression was induced systemically from local inflammation. Epidermal Langerhans cells (skin antigen-presenting cells) from transgenic mice were still immature after DNFB treatment (FIG. 3E). Mixed leukocyte reaction revealed the dysfunction of dendritic cells, but not T cells, in transgenic mice (FIG. 3F). T memory cells from DNFB-sensitized mice were labeled with CFSE, mixed with splenic CD 11c cells pulsed with DNBS, incubated for 60 hrs, and the rate of reduced CFSE intensity was measured. Memory T cells were stimulated less by dendritic cells from transgenic mice than by those from wild type mice. In fact, memory T cells from transgenic mice proliferated equally when they were mixed with dendritic cells from wild type mice.

### Example 4

### Gene Expression Levels of IL-37 in Blood Cells from Normal Subjects and Melanoma Patients

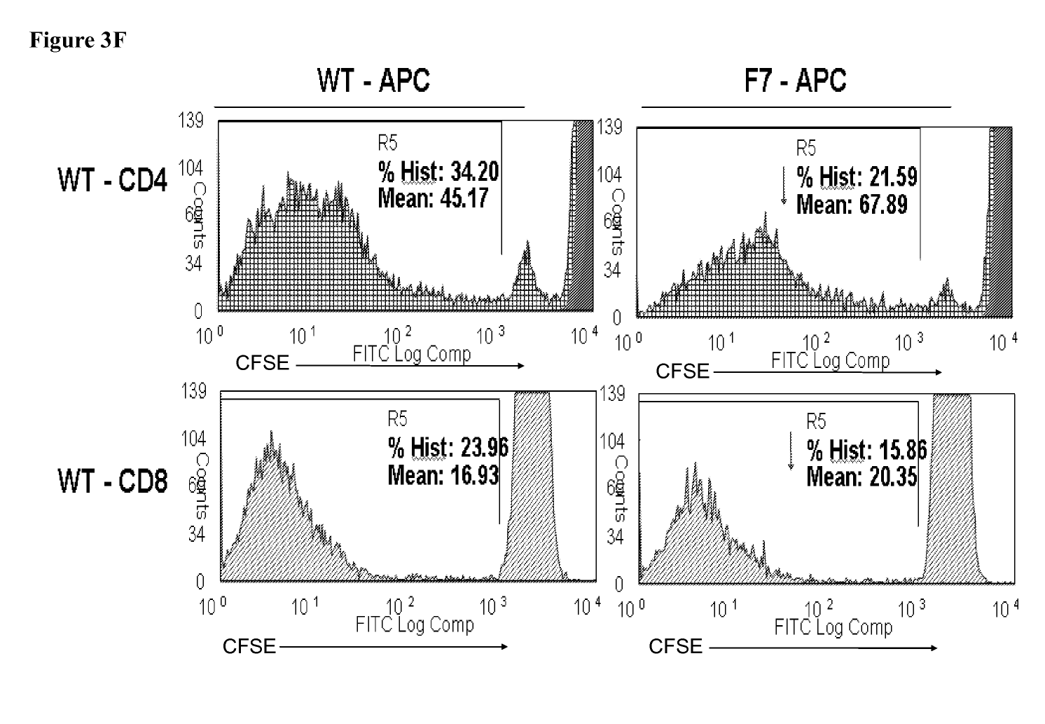

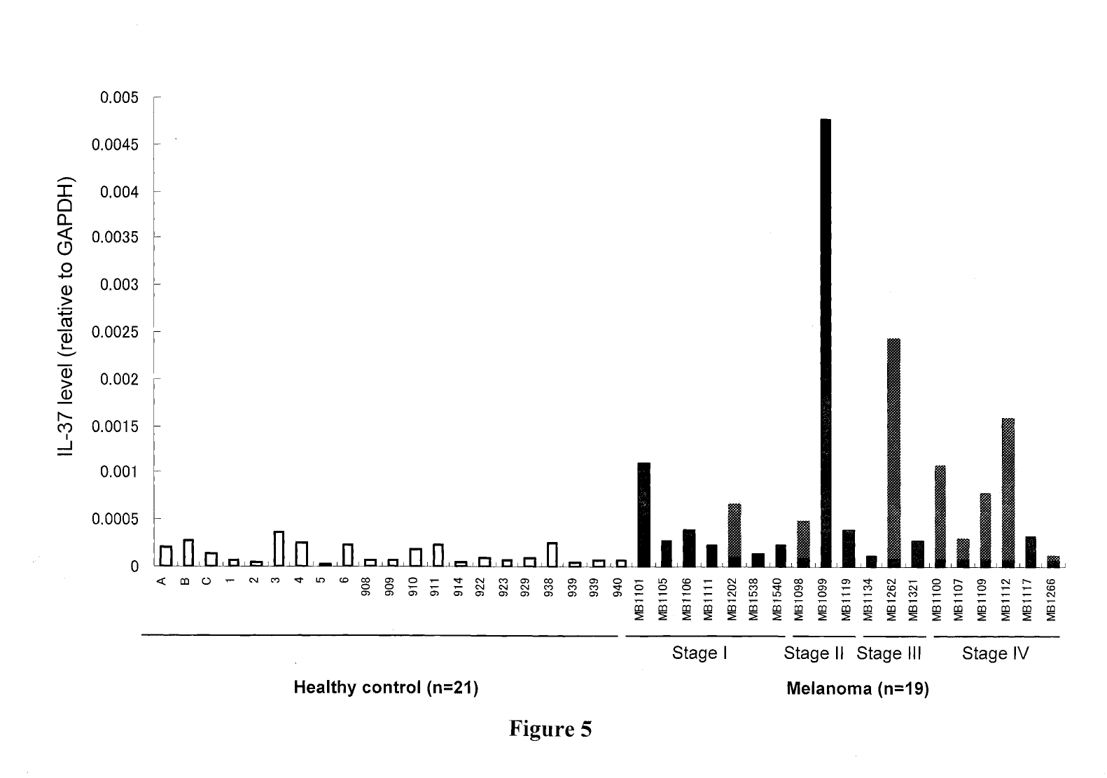

A qRT-PCR analysis of IL-37 gene expression in CD45+ cells in blood samples taken from 21 healthy controls and 19 melanoma patients demonstrated that blood levels were significantly higher in melanoma patients in all stages of melanoma (FIG. 5) and the difference in the IL-37 gene expression levels between healthy controls and melanoma patients was statistically significant (FIG. 6). Expression levels of IL-37 were normalized to the house keeping gene glyceraldehyde 3-phosphate dehydrogenase (GAPDH). Levels of IL-37 expression in healthy controls and melanoma patients were 15.1±1.2×10⁻⁴ and 80.5±12.2×10⁻⁴, respectively (FIG. 6). Table 1 stratifies these data by IL-37 gene expression level, as measured by qRT-PCR in blood samples, to show diagnostic accuracy based on different gene expression level cut off values.

TABLE 1
Expression level cut off and diagnostic accuracy.

| IL-37 | No of | No of | No of | No of | Normal | Melanoma |
|---|---|---|---|---|---|---|
| expression | healthy | healthy | melanoma | melanoma | correct | correct |
| (10-4) | CORRECT | FALSE | CORRECT | FALSE | classification | classification |
| ≦27, >27 | 20 | 1 | 12 | 7 | 95.2% | 63.2% |
| ≦26, >26 | 19 | 2 | 14 | 5 | 90.5% | 73.7% |
| ≦25, >25 | 18 | 3 | 14 | 5 | 85.7% | 73.7% |
| ≦24, >24 | 17 | 4 | 14 | 5 | 81.0% | 73.7% |
| ≦23, >23 | 16 | 5 | 16 | 3 | 76.2% | 84.2% |

For example, IL-37 expression levels above/below 0.00022 will discriminate between the two groups with the following accuracy:

16/19 (84.2%) of melanoma patients show IL-37 >0.00022.

16/21 (76.2%) of healthy controls show IL-37 <0.00022. However, the healthy control levels whose IL-37 levels were over 0.00022 were low compared with melanoma patients (0.00024 to 0.00027, with only one person showing IL-37 of 0.00036).

When stratified by stage of melanoma, IL-37 gene expression from blood samples showed statistically significant differences between healthy controls and melanoma patients from stage I, II, III and IV disease (FIG. 7). Levels of IL-37 in healthy controls, stage I, II, III, IV melanoma patients were 15×10⁻⁴, 43×10⁻⁴, 188×10⁻⁴, 94×10⁻⁴ and 70×10⁻⁴, respectively. Note that even melanoma patients with stage I disease (with melanoma thickness <1 mm) show statistically elevated IL-37 gene expression levels.

### Example 5

### IL-37 Gene Expression Levels Accurately Predict Disease Progression in Melanoma Patients

Fourteen melanoma patients whose blood samples were analyzed for IL-37 gene expression were clinically followed for up to 22 months. Eight patients showed disease progression (for example, stage I/II to stage III or IV; stage III to stage IV; stage IV to death) and 6 patients were stable (no progression to a different stage) (FIG. 8). This demonstrates that the IL-37 gene expression model (IL-37 gene expression level <0.0005 or >0.0005) can predict disease progression from melanoma patients: 66.7% of melanoma patients whose IL-37 gene expression levels were <0.0005 remained stable, 100% of melanoma patients whose IL-37 gene expression levels were >0.0005 showed disease progression.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1-28. (canceled)

29. A method of determining the stage of cancer in a patient comprising:
obtaining a biological sample from a patient that has been diagnosed with melanoma;
determining the level of IL-37 expression in at least one cell in the biological sample obtained from the patient;
comparing the level of IL-37 expression in the at least one cell to the level of IL-37 expression in at least one cell in the biological sample obtained from a subject that does not have a cancer;
selecting the patient having an expression level of IL-37 in the at least one cell in the biological sample that is between 3-fold and 4-fold greater than the expression level of the IL-37 in the least one cell in the biological sample obtained from a subject that does not have a cancer, as a having a Stage I melanoma;
selecting the patient having an expression level of IL-37 in the at least one cell in the biological sample that is between 8-fold and 12-fold greater than the expression level of the IL-37 in the least one cell in the biological sample obtained from a subject that does not have a cancer, as a having a Stage II melanoma;
selecting the patient having an expression level of IL-37 in the at least one cell in the biological sample that is between 5-fold and 6-fold greater than the expression level of the IL-37 in the least one cell in the biological sample obtained from a subject that does not have a cancer, as a having a Stage III melanoma;
selecting the patient having an expression level of IL-37 in the at least one cell in the biological sample that is between 4-fold and 5-fold greater than the expression level of the IL-37 in the least one cell in the biological sample obtained from a subject that does not have a cancer, as a having a Stage IV melanoma.

30. The method of claim 29, wherein the analyzing is conducted in conjunction with at least one of a skin examination, dermoscopy, lymph node examination, chest x-ray, CT scan of the chest, head, abdomen, or pelvis, magnetic resonance imaging (MRI), evaluation of other biomarkers from the subject, clinical testing to rule out concurrent infection in the subject, clinical testing to rule out the presence of an autoimmune disorder in the subject, and/or a serum lactate dehydrogenase blood test.

31. The method claim 29, wherein patient is selected as having a stable cancer when the expression level of IL-37 in the at least one cell in the biological sample is less than 3-fold greater than the expression level of the IL-37 in the least one cell in the biological sample obtained from a subject that does not have a cancer.

32. The method of claim 29, wherein the biological sample is a body fluid.

33. The method of claim 29, wherein the body fluid is selected from the group consisting of blood, cultured whole blood, serum, plasma, cerebrospinal fluid, urine, saliva, cancer tissue, peritoneal fluids, plural fluids, tears, and sweat.

34. The method of claim 29, wherein the level of IL-37 is detected with a reagent that specifically detects the IL-37 protein.

35. The method of claim 34, wherein the reagent is selected from the group consisting of an antibody, an antibody derivative, an antibody fragment, and an aptamer.

36. The method of claim 29, wherein the level of IL-37 in the sample is analyzed with a technique that specifically detects IL-37 gene expression.

37. The method of claim 36, wherein the technique is selected from the group consisting of a microarray analysis and qRT-PCR analysis.

38. A method for monitoring the progression of melanoma in a subject, the method comprising:
a) measuring the expression level of IL-37 in a first biological sample obtained from the subject;
b) measuring the expression level of IL-37 in a second biological sample obtained from the subject;
c) comparing the expression levels of IL-37 measured in the first and second samples obtained from the subject with cancer-negative reference levels of IL-37 marker,
d) selecting the subject having an expression level of IL-37 in the second biological sample that is at least 3-fold greater than the expression level of the IL-37 in the first biological sample, as having a progressively developing melanoma; and,
e) selecting the subject having an expression level of IL-37 in the second biological sample that is less than at least 3-fold greater than the expression level of the IL-37 in the first biological sample, as having a melanoma that is not progressing.

39. The method of claim 38, wherein the first biological sample from the subject is obtained at a time t₀, and the second biological sample from the subject is obtained at a later time t₁.

40. The method of claim 38, wherein the first biological sample and the second biological sample are obtained from the subject more than once over a range of times.

41. The method of claim 38, wherein the subject selected as having a progressively developing melanoma is identified as having a melanoma that is progressing from Stage I, to a higher Stage melanoma.

42. The method of claim 38, wherein the subject selected as having a progressively developing melanoma is identified as having a melanoma that is progressing from Stage II, to a higher Stage melanoma.
